(19) United States
(12) Patent Application Publication
(10) Pub. No.: US 2008/0147019 A1
(43) Pub. Date: Jun. 19, 2008

(54) Antimicrobial component system containing metallic nanoparticles and chitosan and/or its derivatives

(75) Inventors: Xuedong Song, Roswell, GA (US); Bao T. Do, Decatur, GA (US); Robert B. Johnson, Marietta, GA (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/641,637

(22) Filed: Dec. 19, 2006

(51) Int. Cl. A01N 25/08; A01N 59/16; A01N 59/20; A01P 1/00; A61J 15/00; A61M 16/04; A61M 25/00

(52) U.S. Cl. 604/265; 424/618; 424/638; 424/488; 604/270; 128/207.14

(57) ABSTRACT

A material composition, including metallic nanoparticles of silver, silver alloys, or copper, having antimicrobial properties is disclosed. The metallic nanoparticles are embedded or encapsulated in a matrix formed of chitosan or chitosan derivative-based compounds.

### FIELD OF INVENTION

The present invention relates to an antimicrobial composition and its uses. In particular, the invention pertains to a chemical agent that is, in part, composed of a combination or complex of metal-containing nanoparticles within a chitosan-based matrix. The-chemical agent exhibits antimicrobial properties that either kill microorganisms or inhibit their growth on a variety of surfaces for multiple kinds of products.

### BACKGROUND

As evidenced in the market by the presence of numerous materials for eliminating or minimizing human contact with bacteria or fungi, a demand clearly exists for materials and/or processes that either minimize or kill harmful bacteria one may encounter in the environment. Such materials are useful in a variety of applications, such as food preparation and handling, personal hygiene, or household and industrial cleaning, or treatment and prevention in hospitals and clinics, nursing homes, or research settings. In particular, in hospitals and nursing homes antimicrobial materials are needed where people with compromised immune system are especially vulnerable to bacterial infections.

Chitosan is derived from chitin, which is the second most abundant polysaccharide in nature. Chitosan and many of its derivatives have been found to be biodegradable and safe, and they have demonstrated to be useful in a wide variety of applications. Chitosan is the commonly used name for poly-[1-4]-β-D-glucosamine. Chitosan is chemically derived from chitin which is a poly-[1-4]-β-N-acetyl-D-glucosamine, which in turn, is derived from the cell walls of fungi, the shells of insects, and especially crustaceans. Thus, it is can be serviced relatively inexpensively from widely available materials. It is available as an article of commerce from, for example, Biopolymer Engineering, Inc. (St. Paul, Minn.); Biopolymer Technologies, Inc. (Westborough, Mass.); and CarboMer, Inc. (Westborough, Mass.).

Ionic silver (Ag+) is highly antimicrobial with the ability to kill a very broad spectrum of medically relevant bacteria (gram+ and gram−), as well as fungi (molds and yeasts). Ionic silver is also oligodynamic, which means that it is antimicrobial at very low doses, as low as about 0.001-0.05 ppm. Although silver is a heavy metal, at the referenced low concentration amounts, it is nontoxic to human cells and therefore very safe.

Chitosan can be treated with metal salt solutions so that the metal ion forms a complex with the chitosan. Chitosan and chitosan-metal compounds have demonstrated antimicrobial activity (see, e.g., T. L. Vigo, “Antimicrobial Polymers and Fibers: Retrospective and Prospective,” in BIOACTIVE FIBERS AND POLYMERS, J. V. Edwards and T. L. Vigo, eds., ACS Symposium Series 792, pp. 175-200, American Chemical Society, 2001).

### SUMMARY OF THE INVENTION

The present invention discloses compositions that include metal-containing nanoparticles and chitosan and/or chitosan derivatives. The composition may further include one or more other antimicrobial, anti-viral or anti-fungal agents. The anti-viral reagent may be an organic acid, such as acetic, citric, or salicylic acid, with a pH of about 2-6, in an amount from about 0.1 wt. % up to about 3 wt. %, typically about 0.5-2 wt. %. Metal-containing nanoparticles refer to metallic silver nanoparticles, silver-containing alloy nanoparticles, and metallic copper nanoparticles. The compositions can be employed in the form of particles, films, coatings, fibers, or bulk materials. The compositions can be used for inhibiting microorganism growth or killing microorganisms. The present invention relates in part to a composition that includes metal-containing nanoparticles in a chitosan-containing or chitosan derivative-containing matrix as a protective agent or barrier against bacteria. The chitosan-containing matrix functions to encapsulate or stabilize or support (i.e., keep in place) the metallic nanoparticles, and to act as a carrier vehicle for applying the metallic nanoparticles to a substrate or item to be treated with the protective agent. The metal-containing nanoparticles (either on the surface or encapsulated inside the matrix) can generate silver or copper ions upon exposure to environments with oxygen (or other oxidation agents) and water.

The present composition permits one to control the relative level of oxygen access to the surface of the silver metal nanoparticles, and the in situ generation of silver ions. It is believed that the chitosan matrix protects the metallic nanoparticles from uncontrolled oxidation. The chitosan matrix can be in the form of a permeable film. Oxygen is able to diffuse into the chitosan matrix, and upon oxidation of the silver metal nanoparticles, silver ions are generated in the film. Successive layers of nanoparticles of metallic silver, copper or their alloys each within the film the matrix undergo controlled, slow oxidization as a result of exposure to atmospheric, ambient moisture. These surface layers generate in-situ silver or copper ions, which are released slowly into the surrounding environment and have an antimicrobial effect. When bacteria come into contact with the silver or copper ions, they are killed. In another aspect, the chitosan matrix can also function as a protective barrier. The film can be applied to a substrate as a coating layer. The chitosan molecules in the matrix may be adapted or functionatized with organic functional groups, which may facilitate attachment of the chitosan to various materials readily. Alternatively, the referenced metallic nanoparticles also can be encapsulated within a permeable chitosan shell or membrane.

The present invention also relates to a method of generating an antimicrobial agent in-situ. The method entails: providing a material composition composed of a chitosan or chitosan-based derivative compound matrix having a number of silver metal nanoparticles impregnated or encapsulated within the matrix, providing a substrate with a surface; treating the substrate surface with a layer of the material composition; exposing the silver metal nanoparticles to an oxidizing agent to generate silver ions from a surface layer of the metal nanoparticles.

In another aspect, the invention relates to consumer products, healthcare products or medical devices having a protective coating that prevents formation of bio-films. The medical devices that are treated with the material composition comprising a film of chitosan, chitosan-derivative compounds, or a combination of both that forms a supportive or encapsulating matrix for a plurality of metallic silver nanoparticles, said nanoparticles within, said nanoparticles having a particle size in a range from about 5 nm up to about 250 nm, and said chitosan or chitosan-derivative compounds are present in terms of total weight in an amount of at least 50 wt. %, said nanoparticles are present in an amount of about 0.01 wt. % up to about 15 wt. %, 0-10% crosslinking agents, and about 0-30 wt. % of a chemical or physical modifier species or combinations thereof.

Other features and advantages of the present material composition and devices treated with the compositions will become evident from the following detailed description. It is understood that both the foregoing general description and the following detailed description and examples are merely representative of the invention, and are intended to provide an overview for understanding the invention as claimed.

### BRIEF DESCRIPTION OF FIGURES

FIG. 1, is a scanning electron micrograph (SEM) of a thin film layer of chitosan.

FIG. 2, shows a SEM of silver metal nanoparticles (˜80 nm) impregnating and fixed within a film matrix of chitosan.

FIG. 3, shows a SEM image of an aggregate of silver nanoparticles, with the aggregate diameter of about ˜400 nm, in the chitosan matrix. The individual nanoparticles have a diameter of about 60-90 nm, average ˜80 nm.

FIGS. 4A and 4B are culture dishes containing bacterial colonies of P. aeruginosa (PA). The culture dish of FIG. 4A is a control sample, while the culture dish of FIG. 4B contains metallic silver nanoparticles in addition to the chitosan film. The thin film of chitosan acetate alone does not effectively inhibit the growth of PA, while one can observe a clear zone inhibition for chitosan acetate/silver nanoparticle thin film, indicating the effective inhibition of PA growth is present in FIG. 4B.

FIGS. 5A and 5B are culture dishes containing bacterial colonies of S. aureus (SA). Similar to the example in FIGS. 4A and 4B, the thin film of chitosan acetate alone does not inhibit growth of SA in FIG. 5A, while a clear zone of inhibition of SA growth has developed for the chitosan acetate/silver nanoparticle thin film in FIG. 5B.

FIGS. 6A and 6B are culture plates containing bacterial colonies of S. aureus (SA). Similar to the example in FIGS. 5A and 5B, a thin film of chitosan acetate alone does not inhibit growth of SA in FIG. 6A, but a clearly discernable zone of inhibition of SA growth is shown in FIG. 6B, using a chitosan-based matrix film that has been treated with copper nanoparticles.

### DETAILED DESCRIPTION OF THE INVENTION

Chitosan (chitin) is the second most abundant natural polysaccharide after cellulose. Chitosan and its derivatives have recently found a wide variety of commercial applications from inhibition of microorganism growth as food and seed wrappers, water purification, drug delivery/controlled release, food supplement to wound healing. However, its full commercial potential has just started to emerge and many more commercial applications are expected in the near future. Antimicrobial agents, such as antibiotics and silver nanoparticles have been used as effective antimicrobial agents for a broad range of microorganisms. They have been used in a wide variety of products to fight growth of microorganisms. For example, silver nanoparticles are used for wound dressings to prevent infections. In many cases, antimicrobial agents are preferred to be encapsulated in various kinds of matrices that can control the release of the antimicrobial agents or prevent leaching of the antimicrobial agents from certain locations. For instance, antibiotics have been used to be encapsulated in hydrogels as coatings for catheters for bio-film prevention. However, they have only a limited lifetime because of leaching caused by large pores of hydrogels and a limited amount of active agent. For many applications, there still exist many needs that can meet various requirements, particularly for healthcare products or medical devices. This invention discloses material systems that contain chitosan or chitosan derivatives and metal nanoparticles where chitosan or chitosan derivatives act as a matrix for encapsulation of antimicrobial agents.

The present invention describes a material composition system that contains chitosan or chitosan-derivatives as a major component of a matrix for encapsulating or embedding metallic nanoparticles. The chitosan-based matrix functions as a carrier for the metal-containing nanoparticles (metallic silver, silver alloy, or copper). The nanoparticles can have a surface that is either bare or modified for enhancing their active properties or rheology for processing. The nanoparticle surfaces can be modified with organic functional substituents, polymers or plastic oligomers, or carbon powder particles. Nanoparticle research is currently an area of intense scientific research, due to a wide variety of potential applications in biomedical, optical, and electronic fields. The interesting and sometimes unexpected properties of nanoparticles are partly due to the aspects of the surface of the material dominating the properties in lieu of the bulk properties. The properties of materials change as their size approaches the nanoscale. For example, the bending of bulk copper (wire, ribbon, etc.) occurs with movement of copper atoms/clusters at about the 50 nm scale. Copper nanoparticles smaller than 50 nm are considered super hard materials that do not exhibit the same malleability and ductility as bulk copper. The percentage of atoms at the surface of a material becomes significant as the size of that material approaches the nanoscale. For bulk materials larger than one micrometre the percentage of atoms at the surface is minuscule relative to the total number of atoms of the material. Suspensions of nanoparticles are possible because the interaction of the particle surface with the solvent is strong enough to overcome differences in density, which usually result in a material either sinking or floating in a liquid.

Not intended to be bound by theory, it is believed that the metallic nanoparticles embedded within the chitosan-based matrix undergo oxidization of metal atoms on their surfaces and slow release of the oxidized atoms, when exposed to water and oxygen or other oxidation agent. Antimicrobial ionic silver or copper can be generated in-situ. Silver and copper ions are effective as antimicrobial agents.

Chitosan itself is a mild antimicrobial agent under acidic conditions. Hence, it is believed that the surface or chitosan-containing media may have to be at least slightly acidic. Acidic conditions, however, are generally not compatible with most physiological conditions of the body, which tend to have a neutral or slightly alkaline pH. Under neutral and/or basic conditions, chitosan has proven to be not effective against bacterial bio-film growth.

To address this problem and to develop an antimicrobial protective coating that is more compatible with innate biologically conditions of a patient's body, Applicants have incorporated nanoparticles of metallic elements or alloys in the present antimicrobial composition system. The system according to the present invention, involves employing chitosan or chitosan derivatives that act as a material matrix to surround, embed, or encapsulate metallic silver nanoparticles. We have found that the metallic silver or copper-chitsoan material composition can provide both short and long term inhibition of microbial growth. Chitosan and chitosan derivatives provide a number of advantages. For instance, chitosan and its derivatives are biocompatible, biodegradable, and safe for a wide variety of applications. The material can be processed or handled easily for coating or treating a product article. Moreover, chitosan derivatives can form very smooth and transparent films of various thicknesses. They can be easily cross-linked and tailored to form hydrogel layers. Additionally, chitosan derivatives have been proven as a reliable vehicle for controlled release of active agents, such as used for drug encapsulation and delivery. So far, it is believed that the use of chitosan or chitosan derivatives as a matrix for encapsulating metallic silver or copper nanoparticle-based antimicrobial agents have not been described in the scientific literature. Applicant's experiments have demonstrated that silver nanoparticles encapsulated within films composed of chitosan or chitosan-derivatives can be very effective at inhibiting growth of several kinds of microorganisms.

The matrix arrangement of silver nanoparticles in the present invention enables one to use relatively stable metallic silver nanoparticles and generate in-situ silver ions that function as an active antimicrobial agent against bacteria. The nanoparticles in the present compositions are pure silver metal nanoparticles, silver-containing alloys and copper metal nanoparticles. They are not silver-doped nanoparticles, which typically refers to a particle that is comprised of some other material, as a major component, while silver is a minor component in the composition. In contrast, others have proposed previously the use of silver salts, in which the silver is already in ion form, or silver-doped clays or alloy particles (the silver is also already in ion form), which are not on the nanometer scale.

An important distinction of the present invention is, it is believed, that the silver ions are generated in situ upon exposure to oxygen and water. The systems that contain silver compounds such as silver oxide and silver zeolites have the silver oxidized already. The nanoparticles are embedded or encapsulated within the chitosan-based matrix, and do not migrate that easily. Therefore, it is desirable that when using the present composition that one applies an even or uniform distribution of silver metal nanoparticles in the chitosan matrix. This feature becomes an important parameter for overall efficacy for killing bacteria. The specific colloidal and dispersion properties of finer, smaller sized (i.e., nanometer size) particles versus larger particles contributes to a more uniform distribution and different reactive properties. Relatively greater surface area of the nanoparticles can enhance greater exposure to moisture and oxygen in the environment. Metal nanoparticles that have a smaller grain size will allow one to achieve fuller coverage of a treated substrate surface and less unoccupied “white” space between the particles on a coated substrate.

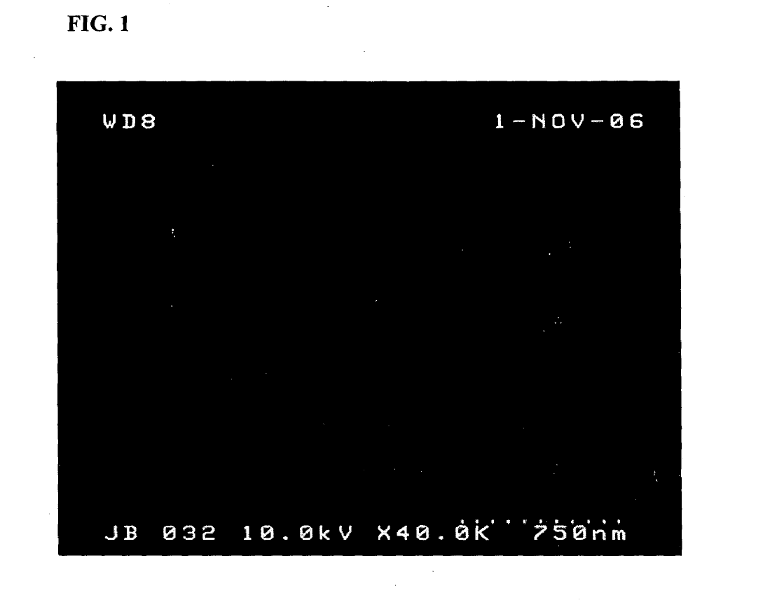

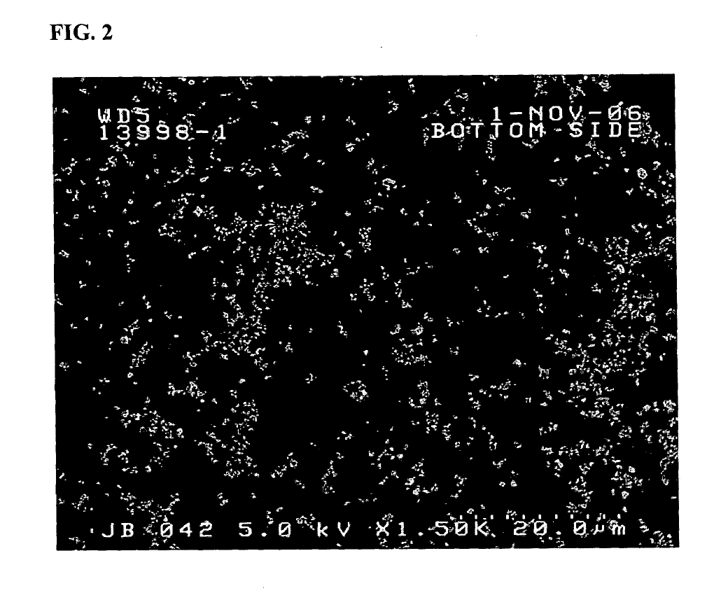

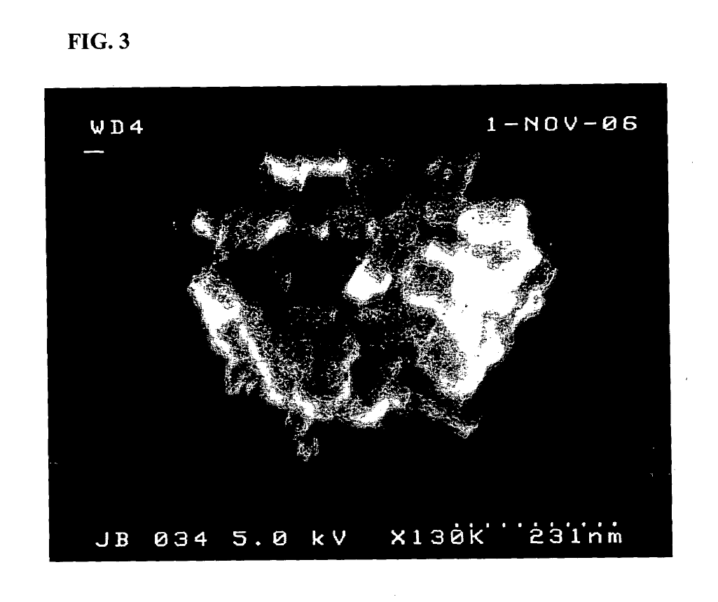

As the accompanying scanning electron micrographs (SEM) show, in FIG. 1, the image of a chitosan clear film without silver nanoparticles, as expected, looks relatively smooth when compared to the image in FIG. 2. The film sample served as a control. FIG. 2 shows a SEM image of a chitosan film having silver metal nanoparticles impregnated or supported in the film matrix. As shown, the particles range in size from about 50-90 nm (average about 80 nm) in diameter. FIG. 3 shows a more detailed, enlarged view of a metal nanoparticles aggregate cluster.

Chemical compounds containing silver ions are also useful as antimicrobials. Typically, however, metallic silver is not an effective antimicrobial agent, whether it is in the form of thin films, nanoparticles, or colloidal silver. However, the precise mode of action of silver salts in killing microbes is yet to be established.

The nanoparticles can have a size that ranges from about 1 or 5 nm to about 200 or 250 nm in diameter, but typically range from about 15-90 or 100 nm, and more typically about 20 or 25-80 nm. Depending on the encapsulation and coating techniques and desired distribution properties, some embodiments may have an average diameter of about 30 to 70 nm, or 40-60 nm. Desirably, the particles have a size of about 35 or 40 nm to about 70 or 85 nm. In certain embodiments, the particle size is desirably under about 60 nm.

The amount of metallic silver, silver alloy or copper nanoparticles incorporated in a chitosan matrix can range from about 0.01% up to about 15% by weight of the chitosan weight. That is, for instance, a sample with 1 wt % silver nanoparticles refers to 1 milligram of silver nanoparticles per 99 miligrams of chitosan. Typically, the amount of silver present is about 1 wt. % up to about 12 wt. %. More desirably, the silver content is about 3-5 wt % up to about 10 wt %.

As envisioned, the present compositions may include a variety of different species as chemical, physical or surface modifiers. The modifiers may include organic species (e.g., mercapto compounds, silyl compounds, urea, TEGO® additives (from Degussa)), or polymeric species (e.g., polyethyleneimine (PEI), poly(methylmethacrylate) (PMMA), polyurethane, polyethylene glycol (PEG), polyvinylpyrrolidone (PVP)), or inorganic species (e.g., montmorillonite (clay), polyhedral oligomeric silsesquioxanes (POSS)). Chitosan derivative compounds may include, but are not limited to, chitosan salts formed with acids, including organic acids (e.g., lipoic acid, citric acid, pyrithione, or oxalic acid), and inorganic acids (e.g., hydrochloric acid, or phosphoric acid), chitosan molecules modified chemically with hydroxyl or amino groups. These derivatives can be either aqueous soluble or insoluble polyelectrolytes or amphiphilic polyelectrolytes, or neutral polymers. Alternatively, the chitosan or chitosan derivatives may include salts, such as quarternized derivates (e.g., trimethylammonium chloride chitosan, NNN-trimethyl chitosan), or organically modified variants (e.g., heptanoyl-chitosan, n-octyl-chitosan). The modifiers may be present individually or in combination, as the properties are desired. The modifiers can be present up to about 60 wt. % of the composition, but more typically is present from about 0 wt % to about 25 or 30 wt %, more typically about 1 wt % to about 10 wt % or 15 wt %.

In certain embodiments, the chitosan matrix may be crosslinked with the metallic silver nanoparticles. A portion of the nanoparticles may be present on an outer surface of the matrix. In other embodiments, the chitosan may be soluble in water.

The antimicrobial agent can be applied in the form of particles, film coatings, or bulk materials. Bulk systems may be in the form of solids, gels, hydrogels, emulsions or suspensions. Given that chitosan is plentiful, relative inexpensive, biodegradable, and non-toxic, it is an ideal material to use to secure the nanoparticles.

Depending on the compositional parameters the chitosan matrix can be either a very or less viscous medium. When the matrix is less viscous, one may need to incorporated cross-linker agents to secure the silver nanoparticles within the chitosan matrix.

Instead of a typical black-colored coating, the relatively small size of the metallic silver nanoparticles ensures optical or light transparency. The silver particles are present in a microscopic layer, and are more uniform or evenly distributed than previously observed over a coated surface. These features are advantages of the present composition.

Both organic and inorganic species of antimicrobial reagent precursors can be easily transformed into active antimicrobial agents. Examples of precursors can include organic reagents, such as, clavulanic acid ester; aminoglycosides amide; dehydroacetic acid ester; sorbic acid ester. These organic precursors can be easily transformed into their corresponding active acid forms by acid, base and enzyme-catalyzed hydrolysis. Inorganic reagents may include: copper oxide; copper hydroxide; or titanium dioxide. These compounds can be transformed into active form in the presence of acid and water.

The chitosan and chitosan derivatives acting as a matrix for encapsulation of metal nanoparticles may or may not be released from the matrix. The metal nanoparticles may be physically encased, embedded or otherwise physically secured inside the chitosan matrix or chemically bonded with the chitosan matrix for surface-modified metal nanoparticles. In some cases, covalent linkages may be desirable, depending on the particular use, although some ionic interactions may also occur. A portion of the metal nanoparticles may or may not be present on the surfaces of the chitosan film or coating. In another embodiment, a system may contain one or more different chitosan derivatives and one or more different metal nanoparticle, where the chitosan derivatives constitute the majority components in the material system. In some embodiments, depending on the desired processing parameters or application to particular substrates or uses, the chitosan derivatives may or may not be water soluble. The chitosan derivative may or may not be cross-linked.

The film may be coated on solid substrates. Another embodiment of the invention is particles that contain the systems described above. The particles may have different shapes and geometries.

Another embodiment of the invention is bulk materials that contain the systems described above. The bulk materials may be in a form of solid, gel, hydrogel, suspension or emulsion. Another embodiment of the invention is a system that contains the chitosan and/or chitosan derivatives and metal nanoparticles plus other ingredients. Embodiments of the chitosan derivatives include, but not limited to, chitosan salts formed with acids, including organic acids such as lipoic acid, vitamin C, pyrithione, oxalic acid, and/or inorganic acids such as hydrochloride acid and phosphoric acid.

In other embodiments, the chitosan derivatives may also include chitosans that are modified chemically with hydroxy groups or amino groups. The derivatives may be water soluble or insoluble. Derivatives may be a polyelectrolytes or amphiliphilic polylectyrolytes, or neutral polymers. The embodiment of the metallic nanoparticles may include metallic copper or metallic silver and silver-containing alloys. Embodiments of microorganisms include, but not limited to, bacteria, viruses, yeasts, spores, and molds.

One embodiment of the applications of the system is to form a film or coating on the surface of medical devices to prevent the development of bio-film growth. Examples of the medical devices may include catheters, feeding tubes, implanted metal or plastic devices and tracheal tubes.

The present metallic nanoparticle-chitosan/chitosan-derivative systems can be fabricated into film or coatings on the surface of solid substrates according to innovative methods or procedures. One example of a method involves formation of a solution or suspension of the system followed by casting a film followed by drying. A neutral film is formed by neutralizing the film by a basic agent if the chitosan is a salt with an acid. Another embodiment of the invention is a nonwoven material or woven material or gauze or dressing that contains the disclosed material systems. Another embodiment of the invention is a gel or hydrogel that is formed from the system. The chitosan/chitosan derivatives may or may not be cross-linked.

It is also envisioned, that the silver nanoparticles containing chitosan and its derivatives can be used to promote wound healing with minimal antimicrobial growth, as part of the bandages or other dressings as an alternative embodiment. For such applications, the present nanoparticles are durably adhered to the chitosan film, hence the nanoparticles will not escape and possibly contaminate a patient's open wounds.

### Section II—Empiricals

### EXAMPLE 1

Preparation of chitosan derivative: 1 g chitosan, low molecular weight from Aldrich, and 1 g pyrithione, from Aldrich, were mixed in 50 ml water. The materials were completely dissolved in the bath and sonicated for about 15 minutes. The solution was dialyzed three times using dialysis tubes that were obtained from Pierce Biotechnology Inc with cut-off MW of 3,500. The chitosan derivative was designated as CTX-P.

### EXAMPLE 2

A chitosan derivative may be prepared according to the following: Using about 1.5 g chitosan, low molecular weight from Aldrich, and 2 ml of acetic acid were mixed in 100 ml water, the materials were completely dissolved in an aqueous bath by sonication for 15 minutes. The solution was dialyzed three times using dialysis tubes that were obtained from Pierce Biotechnology Inc with cut-off MW of 3,500. The chitosan derivative was designated as CTX-Ac.

### EXAMPLE 3

Preparation of film containing CTX-Ac/silver: 6 ml of CTX-Ac was added with 18 ul of silver nanoparticle (25 nm, 50 mg/ml from Novacentrix (Austin, Tex.), in ethanol). The silver nanoparticles were suspended in an aqueous medium while sonicating the mixture for 1 hr followed by vortexing for 10 minutes. The mixture was then loaded to a petri dish. The dish was put in a hood to allow air dry overnight. A thin and transparent film was formed at the bottom of the petri dish and can be peeled off easily. The film was insoluble in acetone and ethanol, but become soft in water.

### EXAMPLE 4

Preparation of film containing CTX-silver: 6 ml of CTX-Ac was added with 18 μl of silver nanoparticle (25 nm, 50 mg/ml from Novacentrix (Austin, Tex.), in ethanol). The silver nanoparticles were suspended by bath sonicating the mixture for 1 hr. followed by stirring in a vortex for about 10 minutes. The mixture was then loaded to a petri dish. The dish was put in a hood to allow it to air dry overnight. The bottom of the petri dish was then exposed to the vapor of an ammonium hydroxide solution overnight. A thin and transparent film was formed at the bottom of the petri dish and the film was then dried at 40° C. for one hour. The film can be peeled off easily. The film was insoluble in acetone, ethanol and water.

### EXAMPLE 5

Preparation of film containing CTX only: 6 ml of CTX-Ac was then loaded to a petri dish. The dish was put in a hood to allow drying in air overnight. The bottom of the petri dish was then exposed to the vapor of an ammonium hydroxide solution overnight. A thin and transparent film was formed at the bottom of the petri dish and the film was then dried at 40° C. for one hour. The film can be peeled off easily. The film was insoluble in acetone, ethanol, and water.

### EXAMPLE 6

As one can observe from the accompanying scanning electron microscopy (SEM) images, in FIGS. 1-3, the metallic nanoparticles have a relatively even or uniform distribution pattern over the chitosan-based film matrix. Taken using a Hitachi S-4500 field emission instrument, the images were acquired using the upper secondary electron detector (high resolution imaging mode). Accelerating voltages of 1.2 and 5.0 kV were used to image completely uncoated samples. In FIG. 2, the chitosan film is impregnated with silver nanoparticles of about 80 nm size. The image shows dispersion of nanoparticles throughout the chitosan film. The relative small size of the nanoparticles allows for more complete coverage within the chitosan matrix than what would be expected of larger particles of silver.

### EXAMPLE 7

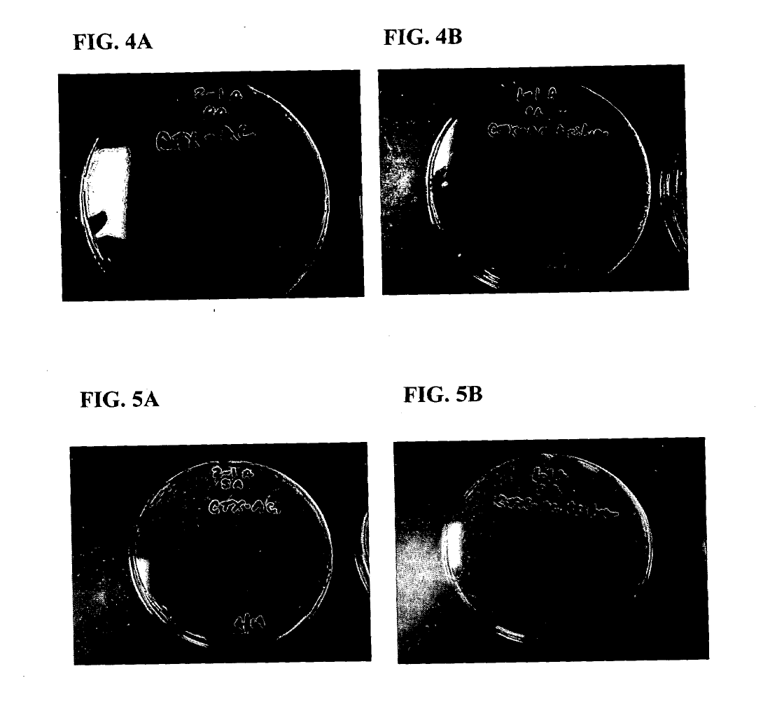

The inhibition of microorganism growth on metallic nanoparticles-treated chitosans films is illustrated with the following example: The films of CTX with and without silver were cut into small pieces and put at the center of augar plate inoculated with P. aeruginosa (ATCC #9027). The P. aeruginosa was allowed to grow at 35° C. overnight. It was found that CTX film with silver can inhibit the growth of P. aeruginosa while CTX films without silver don't inhibit the growth of P. aeruginosa. The accompanying FIGS. 4A and 4B show that thin films of chitosan derivative alone are not effective antimicrobial agents under physiological conditions. FIG. 4A is a control sample containing a square sample of chitosan-based film in a culture dish. As one can see, the bacteria have covered the entire growth surface of the dish. The edge of the chitosan film is barely discernable from the surrounding field, which signifies that there is minimal antimicrobial activity. In contrast, one can see a clearly delineated ring or zone of bacterial inhibition around the chitosan film sample in FIG. 4B. FIG. 4B contains a chitosan film matrix similar to that of FIG. 4A, except that the matrix incorporates about 1 wt. % metallic silver nanoparticles. The antimicrobial properties appear to be effective in areas immediately surrounding the nanoparticles coated regions of the surface.

### EXAMPLE 8

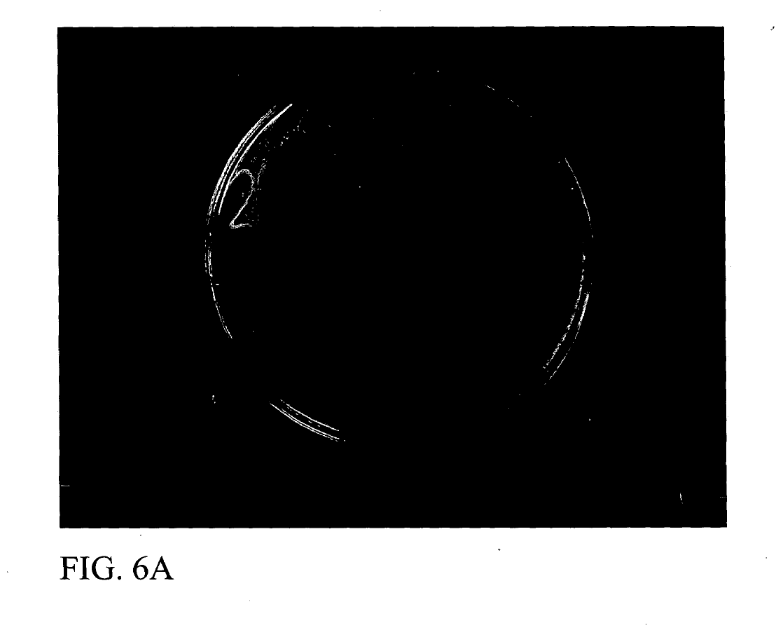

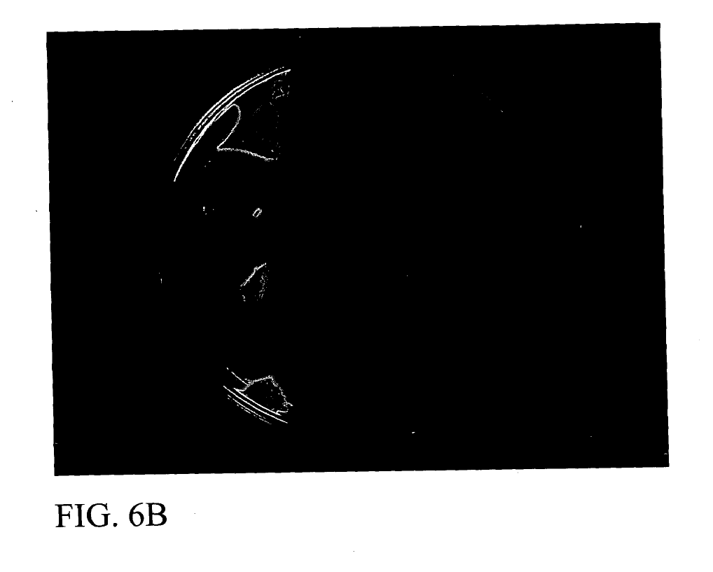

In another example, the chitosan-based nanoparticles composition can inhibit growth of gram negative bacteria, such as S. aureus. The films of chitosan (CTX) with and without silver were cut into small pieces and put at the center of augar plate inoculated with S. aureus (ATCC #6538). The S. aureus was allowed to grow at 35° C. overnight. It was found that a metallic silver nanoparticle-treated CTX film can inhibit the growth of S. aureus while CTX films without silver do not. The accompanying FIGS. 5A and 5B show that thin films of chitosan derivative alone are not effective antimicrobial agents under physiological conditions. FIG. 5A is a control sample containing a square sample of chitosan-based film in a culture dish. As one can see, the bacteria have covered the entire growth surface of the dish. The edge of the chitosan film is barely discernable from the surrounding field, which signifies that there is minimal antimicrobial activity. In contrast, one can see a clearly delineated ring or zone of bacterial inhibition around the chitosan film sample in FIG. 5B. FIG. 5B contains a chitosan film matrix similar to that of FIG. 5A, except that the matrix incorporates about 1 wt. % metallic silver or silver alloy nanoparticles. Similarly, FIGS. 6A and 6B demonstrate the effectiveness of copper nanoparticles in a chitosan-based matrix as an inhibitor of S. aureus colony growth.

The present invention has been described in detail by way of examples. Persons skilled in the art, however, may appreciate that modifications and variations may be made to the present system, compositions and devices without departing from the scope of the invention, as defined by the appended claims and their equivalents.

We claim:

1. An antimicrobial composition comprising: a plurality of metallic nanoparticles having a particle size in a range from about 1 nm up to about 250 nm, in a range of about 0.01 wt. % up to about 15 wt. %, embedded or encapsulated within a matrix containing by total weight at least 10% of chitosan or chitosan-derivative compounds, 0-10% crosslinking agents, and up to about 60% of chemical or physical modifier agents.

2. The antimicrobial complex according to claim 1, wherein said metallic nanoparticles are formed of either metallic silver, silver alloy, metallic copper, or a combination thereof.

3. The antimicrobial composition according to claim 2, wherein said metallic nanoparticles have a surface that is modified to change either its chemical or physical properties.

4. The antimicrobial composition according to claim 1, wherein said modifier agent enhances crosslinkages and stabilizes said nanoparticle within said matrix.

5. The antimicrobial composition according to claim 1, wherein said metallic silver nanoparticles have a mean particle size of about 15 nm to about 100 nm.

6. The antimicrobial composition according to claim 1, wherein said metallic nanoparticles are present from about 0.5 wt. % to about 12 wt. %.

7. The antimicrobial composition according to claim 1, wherein said chitosan matrix is a carrier vehicle for said nanoparticles.

8. The antimicrobial composition according to claim 1, wherein said chitosan matrix forms a thin film, membrane, or shell that surrounds said metallic nanoparticles.

9. The antimicrobial composition according to claim 1, wherein said chitosan or chitosan-derivative compounds are present up to about 90 wt. %.

10. The antimicrobial composition according to claim 1, wherein said chitosan or chitosan-derivative compounds are present from 15-80% by weight.

11. The antimicrobial composition according to claim 1, wherein said chemical or physical modifier includes organic, inorganic or polymeric species.

12. The antimicrobial composition according to claim 1, wherein said chemical or physical modifier are present in an amount of about 1-30 wt. %.

13. The antimicrobial composition according to claim 1, wherein said modifier agents include organic, inorganic, or polymeric species that modify either the chemical or physical properties of the surface of said metallic nanoparticles, said chitosan or chitosan-based derivative compound matrix, or both.

14. The antimicrobial composition according to claim 1, wherein said modifier agents adjust durability, elasticity, softness, and wettability properties of said matrix.

15. The antimicrobial composition according to claim 1, wherein said modifier agents positively impact solubility, and chemical functionality of said nanoparticles and matrix, or the relative bonding of said composition to a substrate surface.

16. The antimicrobial composition according to claim 1, further comprising a second antimicrobial agent including anti-biotic, anti-viral, anti-fungal, and anti-yeast reagents, either individually or in combination.

17. A method of generating an antimicrobial agent in-situ, the method comprises: providing material composition composed of a chitosan or chitosan-based derivative compound matrix having a number of metallic nanoparticles impregnated or encapsulated within said matrix; providing a substrate surface; treating said substrate surface with a layer of said material composition; exposing said silver metal nanoparticles to an oxidizing agent to generate silver ions from a surface layer of said metallic nanoparticles.

18. The method according to claim 17, wherein said chitosan matrix forms a gas permeable film or membrane.

19. The method according to claim 17, further comprises killing bacteria that are present on said treated substrate surface and immediate surrounding areas.

20. The method according to claim 17, wherein said metallic nanoparticles are selected from metallic silver, silver alloys, or metallic copper.

21. A medical device having a protective coating that prevents formation of bio-films, said coating comprising a film of chitosan, chitosan-derivative compounds, or a combination of both that forms a supportive or encapsulating matrix for a plurality of metallic silver nanoparticles, said nanoparticles within, said nanoparticles having a particle size in a range from about 1 nm up to about 200 nm, and said chitosan or chitosan-derivative compounds are present in terms of total weight in an amount of at least 50 wt. %, said nanoparticles are present in an amount of about 0.01 wt. % up to about 15 wt. %, 0-10% crosslinking agents, and 0-25% organic, inorganic or polymeric surface modifiers.

22. The medical device according to claim 21, wherein said medical device is one of the following:
catheters, feeding tubes, implanted metal or plastic devices, and tracheal tubes.

23. A controlled-release antimicrobial agent system comprising: a plurality of metallic nanoparticles embedded or encapsulated within a chitosan or chitosan-derivative-based matrix, said nanoparticles being composed of metallic silver, silver alloys, metallic copper, or combinations thereof, and having a mean particle size in a range from about 1 nm up to about 200 nm, in a range of about 0.01 wt. % up to about 15 wt. % by total weight, said matrix containing about 10% to 50% of chitosan or chitosan-derivative compounds, 0-10% crosslinking agents, and 0-40% of an agent that modifies either a chemical or physical property of either said matrix or surface chemistry of said nanoparticles.
